(19) United States
(12) Patent Application Publication
(10) Pub. No.: US 2010/0197965 A1
(43) Pub. Date: Aug. 5, 2010

(54) METHOD FOR THE CATALYTIC CONVERSION OF 2-HYDROXY-4-METHYLTHIOBUTANENITRILE (HMTBN) INTO 2-HYDROXY-4-METHYLTHIOBUTANAMIDE (HMTBM)

(75) Inventors: Virginie Belliere-Baca, Andressy (FR); Jean-Claude Kiefer, Blaincourt Les Precy (FR); Jean-Christophe Rossi, Villeneuve Les Maguelone (FR)

Correspondence Address: CANTOR COLBURN, LLP, 20 Church Street, 22nd Floor, Hartford, CT, 06103, US

(73) Assignee: ADISSEO IRELAND LIMITED, Dublin 1 (IE)

(21) Appl. No.: 12/671,361

(22) PCT Filed: Jul. 30, 2008
(86) PCT No.: PCT/FR2008/051432

§ 371 Date: Jan. 29, 2010

(30) Foreign Application Priority Data
Jul. 31, 2007 (FR) 0705592

(51) Int. Cl. C07C 51/06; C07C 231/06

(52) U.S. Cl. 562/606; 564/126; 564/127

(57) ABSTRACT

This process is carried out in the presence of a solid catalyst comprising an active phase. The catalyst is formulated and the conversion is carried out in a medium essentially free of strong mineral acid.

### TECHNICAL FIELD

The disclosure relates to the catalytic conversion of 2-hydroxy-4-methylthiobutanenitrile (HMTBN) to 2-hydroxy-4-methylthiobutanamide (HMTBM), illustrated below.

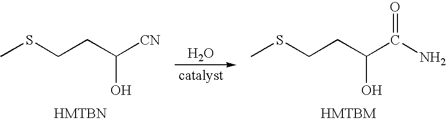

The HMTBM thus obtained can be used, for example, for the production of 2-hydroxy-4-methylthiobutanoic acid (HMTBA), a hydroxy analogue of methionine, methionine being an essential amino acid widely used as a food additive in animal nutrition.

### BACKGROUND

A large number of documents describe the catalytic conversion of 2-hydroxy-4-methylthiobutyronitrile (HMTBN) to 2-hydroxy-4-methylthiobutyramide (HMTBM) and/or 2-hydroxy-4-methylthiobutanoic acid (HMTBA).

Thus, this conversion has been described in the stoichiometric or super-stoichiometric presence of strong mineral acids, such as sulphuric acid. The major drawback of the use of strong mineral acids is their high catalytic activities which do not make it possible to control the selectivity for HMTBM, leading, in addition, to the coproduction of a very large amount of inorganic products that are not easy to exploit. Specifically, the catalytic activity of strong mineral acids with respect to HMTBN is such that all the HMTBN introduced is converted very rapidly. The HMTBM produced can in particular react with water to form HMTBA and aqueous ammonia. In the case of sulphuric acid, for example, it may react with the aqueous ammonia released so as to form ammonium sulphate, which will then have to be processed.

For environmental reasons, one of the proposed alternatives to this acid hydration is an enzymatic process in which a nitrile hydratase, such as Rhodococcus (according to U.S. Pat. No. 6,900,037 B2 and WO 2002/070717 A2, for example), can convert HMTBN to HMTBM. The major drawback which puts this process at a disadvantage lies in the difficulty in synthesizing enzymes and then in extracting them from the reaction medium after the HMTBM has been obtained. A solution provided by patent WO 2002/00869 for extracting the enzymes from the reaction medium is the use of water-insoluble granules containing the enzyme. However, the process for obtaining these granules is complicated and the catalytic activity of the enzymes in the granules is very greatly reduced. The weak catalytic activity makes it possible to be very selective for HMTBM, but the duration of the reaction for conversion of HMTBN to HMTBM must be extended.

Processes by heterogeneous catalysis are also known. According to U.S. Pat. No. 5,386,056, the hydration of HMTBN is carried out in the presence of a manganese oxide-based bulk catalyst in a water/acetone mixture (450/150). The amount of manganese oxide used is 0.75 mol per 1 mol of cyanohydrin. It has become apparent that this reaction is difficult to reproduce since it is strongly linked to the nature of the manganese oxide used. For example, in the presence of pyrolusite, no hydration reaction is apparent. In addition, the conditions for the hydration of HMTBN according to this patent do not appear to be easy to optimize in order to obtain a reproducible yield in the absence of by-products.

Patent FR 2 750 987 describes the reaction for hydration of HMTBN or of methylpropioaminonitrile cyanohydrin to corresponding amide at low temperature (between 0° C. and 60° C.) in water in the presence of a silica-supported manganese oxide-based catalyst, without any appearance of products from oxidation on the sulphur atom. According to this patent, the MnO₂/HMTBN or MnO₂/methylthiopropioaminonitrile cyanohydrin molar ratio is between 0.05 and 1.5. The weight ratio of manganese oxide to silica is preferably between 5% and 10%. It appears, in the examples of this patent FR 2 750 987, that supporting the manganese oxide on silica makes it possible to improve the selectivity of the reaction, but the small amounts of active phase deposited mean that long reaction times and/or very low cyanohydrin concentrations are necessary.

It is known to those skilled in the art that the amount of water used in the reaction medium is not essential for the reaction, but that, if the reaction time is too long and/or the temperature is too high, some of the HMTBM formed can react with the water of the reaction medium so as to form HMTBA and/or to produce HMTBM condensation by-products. Furthermore, in the case of the formation of HMTBA, the aqueous ammonia released induces an increase in the hydrogen potential (pH) of the reaction medium which, at a basic pH, causes decomposition of the HMTBN that has not yet reacted and, consequently, an overall decrease in the production of HMTBM.

Thus, in patent EP 0 601 195 A1, which describes a process for producing HMTBA in three successive steps, the first of which comprises the catalytic conversion of HMTBN to HMTBM in the presence of a heterogeneous catalyst, preferably manganese oxide or an alkali metal borate (sodium tetraborate), it is recommended to substitute part of the amount of water of the reaction medium with a water-soluble organic solvent, such as acetone or methanol, and to add sulphuric acid in order to improve the selectivity of the reaction. According to this patent, sulphuric acid is added so as to improve the performance levels of the reaction, but in very limited amounts in order to avoid the formation of aqueous ammonia. In the examples, the best yield of HMTBM obtained after 6 hours of reaction at 60° C. in the presence of manganese oxide and sulphuric acid in a water/acetone solvent is 89%.

In summary, the art shows that it is difficult to reconcile a strong catalytic activity for the hydration of HMTBN and good selectivity for HMTBM. The best performance levels are described for manganese oxide-based catalysts. In particular, the best selectivities are obtained for silica-supported manganese oxide. The low contents of supported manganese oxides imply long reaction times or reaction media containing very low concentrations of HMTBN.

### BRIEF SUMMARY

The seeks to provide an alternative to the processes described above, but which does not have the drawbacks thereof.

Thus, disclosure provides a solid catalyst that is sufficiently active and selective to produce 2-hydroxy-4-methylthiobutanamide (HMTBM) from 2-hydroxy-4-methylthiobutanenitrile (HMTBN) in short reaction times so as to limit the formation of unwanted by-products and without the addition of strong mineral acids so as not to produce inorganic waste and so as to prevent decomposition of the HMTBN.

The authors of the disclosure have discovered that formulating an active phase for the selective hydration of HMTBN to HMTBM makes it possible to render the solid catalyst active under given conditions making it possible to limit reaction times and to improve, in addition, the selectivity of the reaction. The formulating of the catalyst is carried out in a diluent.

It has also been found that formulating the active phase for the selective hydration of HMTBN to HMTBM in a diluent makes it possible to increase the amount of active phase accessible and effective for the reaction and makes it possible to improve the mechanical strength properties of the catalyst.

Reinforcing the mechanical strength of the catalyst makes it possible to limit the loss of activity of the catalyst over time due to lixiviation of the active phase of the catalyst in the reaction medium. The present invention therefore also makes it possible to improve the lifetime of the catalyst.

Finally, another advantage of having a formulated solid catalyst makes it possible to carry out the reaction in a continuous reactor. The reaction time may then be very readily controlled. The separation of the catalyst from the reaction medium is facilitated. Regeneration of the catalyst may also be envisaged directly in the reactor under a stream of air at temperature, for example.

### DETAILED DESCRIPTION

Thus, a first subject of the invention is a process for the catalytic conversion of HMTBN to HMTBM, in the presence of a solid catalyst comprising an active phase, said catalyst being formulated and said conversion being carried out in a medium essentially free of strong mineral acid.

The term “essentially free of strong mineral acid” is intended to mean a presence, if there is one, in trace amounts at most, i.e. a proportion of less than 0.1% by weight relative to the total weight of the medium.

According to this invention, the active phase for the selective hydration of HMTBN to HMTBM includes at least one metal oxide. The proportion of this active phase is preferably at least 30% by weight relative to the total weight of the catalyst.

The metal elements constituting these oxides are advantageously chosen from the group comprising copper, nickel, iron, zirconium, manganese and cerium, and combinations thereof. The preferred metal oxides are manganese oxide and cerium oxide; they may be present alone or in combination so as to promote the selective hydration of HMTBN to HMTBM.

According to another feature of the invention, the diluent is chosen from the group comprising zirconium oxide, titanium oxide, alumina, silica, clays such as bentonites, and attapulgite, and combinations thereof. The proportion of said diluent is preferably at most 70% by weight relative to the total weight of the catalyst. As preferred diluent of the invention, mention may be made of silica and alumina, and combinations thereof.

The formulating of the catalyst comprises, in general, at least a first step of formulating the active phase, followed by a second step of heat treatment. As an example of a formulation process, mention may be made of the processes using wet granulation or extrusion, in the presence of a binder. The heat treatment step is commonly a drying step: (low temperature) between 50° C. and 100° C., followed by a calcination step, the objective of which is to reveal the active phase, between 200° C. and 600° C.

The term “binder” is intended to mean any binder chosen from water, natural polymers, organic polymers and sugars, characterized in that it will make it possible to ensure cohesion of the active phase and of the diluent during the preparation of the catalyst.

The term “natural polymer” is intended to mean any natural polymer, for instance starch, gelatine, alginic acid or sodium alginate, and combinations thereof.

The term “organic polymer” is intended to mean any organic polymer, for instance polyvinylpyrrolidone, methylcellulose or polyethylene glycol, and combinations thereof.

The term “sugar” is intended to mean any sugar, for instance glucose, sucrose or sorbitol, and combinations thereof.

This list of binders is given by way of indication and is not exhaustive. Thus, any binder which makes it possible to improve certain properties of the invention is suitable, binders which do not generate toxic compounds or which are not themselves toxic to the environment or to the catalytic reaction being preferred.

A first embodiment of the process for obtaining these compositions by granulation comprises the following steps:
a mixture of the powders of active phase and of diluent, the proportions of which are determined by the desired composition of the catalyst formulated, is prepared;
granules of small sizes (<1 mm) of desired formulation are generated, and are called initiators;
a dilute solution of binder is prepared;
the initiators are introduced into the granulating dish, also called pan granulator, the mixture of active phase and diluent powders previously prepared are continuously added slowly to said initiators, and the binder solution is simultaneously sprayed;
granules which are “selected naturally by centrifugation” are produced, said granules being removed from the dish as soon as the desired particle size is reached, via the speed of rotation and the incline of the dish;
the granules are dried and calcined.

A second embodiment of the process for obtaining these compositions by mixer granulation—low or high shear granulator, is used.

These mixers are equipped with one or more rotors of blade, pin or ploughshare type, which make(s) it possible to move the pulverulent mixture. This embodiment comprises the following steps:
a mixture of the powders of active phase and of diluent, the proportions of which are determined by the desired composition of the catalyst formulated, is prepared;
the binder in the form of a spray is incorporated, thereby making it possible to ensure the growth of the granules and to control the particle size distribution by controlling the amount of binder introduced; the other important granulation parameters are the rotation speed and contact time parameters.

The granules with or without subsequent spheronization treatment are subsequently dried and calcined.

A third embodiment of the process for obtaining these compositions by extrusion comprises the following steps:
a mixture of the powders of active phase and of diluent, the proportions of which are determined by the desired composition of the catalyst formulated, is prepared;
the binder is introduced;
the mixture is kneaded until a paste is obtained;
the paste thus obtained is introduced into a die of desired diameter;
the solids of desired diameter are recovered and are cut into the desired object length;
extruded materials are obtained;
the extruded materials are dried and calcined.

The extruded materials can be produced continuously with an extruder into which the mixture of powders, i.e. active phases and diluents, and then the binder are introduced. A paste is thus generated in situ, in the screw, for example single or double screw, and then extracted in the form of “spaghetti strings”, the length of which is controlled by the formulation or mechanically, for example with a rotary knife. They are subsequently dried and then calcined.

A catalyst of the invention exhibits strong activities for the very highly selective hydration of HMTBN to HMTBM at temperatures of between 0° C. and 100° C., more particularly between 20° C. and 90° C.

The reaction time is advantageously greater than 45 minutes, and preferably greater than 60 minutes.

The catalytic hydration of HMTBN to HMTBM can be carried out in the liquid phase or in the gas phase.

Under these conditions, the HMTBN is in solution, at 20% to 80% relative to the total weight of the solution. It may be in solution in a solvent or a mixture of solvents chosen from water and water-soluble solvents such as acetone or methanol.

According to a variant of the process of the invention, the HMTBN is present in a reaction medium from which it originates. It may, for example, be obtained by reaction of hydrogen cyanide with 3-(methylthio)propionaldehyde (MTPA), or else from acrolein and hydrogen cyanide, without the isolation of intermediate products, and then the addition of methylmercaptan (MSH).

The catalytic hydration of HMTBN to HMTBM can be carried out in a closed reactor or continuously. Industrially, the reaction can be carried out in a continuous reactor on a fixed bed of catalyst or in a perfectly stirred reactor, and in particular a continuous reaction on a fixed bed of catalyst is preferred.

As mentioned above, the process of the invention can advantageously be used in the preparation of 2-hydroxy-4-methylthiobutanoic acid (HMTBA), according to the following steps:
the conversion of HMTBN to HMTBM is carried out via a process of the invention as defined above,
the conversion of the HMTBM to HMTBA is carried out.

The step for conversion of the HMTBM to HMTBA can be carried out under conditions well known to those skilled in the art.

Thus, this step can be carried out catalytically, in the presence of a catalyst based on one or more metal oxides, preferably chosen from titanium dioxide and zirconium dioxide.

This conversion step can also be performed by hydrolysis in the presence of an acid, such as a mineral acid chosen from H₂SO₄, H₃PO₄ and HCl. By way of example, the acid is H₂SO₄, and the reaction conditions are those described in application EP-A-1 097 130.

HMTBA can also be prepared from HMTBM enzymatically, in the presence of an amidase.

When it is obtained in the form of an ammonium salt (HMTBS), the ammonium salts optionally as a mixture with the HMTBA are subjected to a conversion treatment, advantageously chosen from neutralization, electrodialysis and distillation. The neutralization step can be carried out on resins, or by acid neutralization.

The aim of the examples which follow is to illustrate the present invention without limiting the scope thereof.

### Example 1

### Preparation of a Catalyst A

A formulated catalyst having a composition of 90% by weight of cerium oxide and 10% by weight of alumina is prepared by wet granulation.

To prepare this catalyst, a cerium oxide from Rhodia, HSA-5, and an alumina SB3 from Condea and water as binder are used.

A mixture of powders composed of 90% by weight of cerium oxide and 10% by weight of alumina is prepared. 10% by weight of initiators for this composition are prepared in a granulating dish. The mixture of powders is then continuously introduced slowly, and the water is simultaneously sprayed in order for the granulation to be effective. The granules produced are “selected naturally by centrifugation”, said granules being removed from the dish as soon as the particle size is reached (4-5 mm), via the speed of rotation and incline of the dish.

They are recovered, dried in an oven for 12 h at 60° C. and then calcined for 2 h at 500° C.

### Example 2

### Preparation of a Catalyst B

A formulated catalyst having a composition of 90% by weight of alpha manganese oxide and 10% by weight of alumina is prepared by extrusion.

To prepare this catalyst, an HSA alpha manganese oxide from Comilog (batch no. 103514-12) and an alumina SB3 from Condea are used.

The “90% by weight of alpha manganese oxide” and “10% by weight of alumina” powders are mixed.

67 g of a mixture of powders are introduced into a Brabender kneading machine and 32 ml of purified water are introduced over 8 minutes. The kneading time after the introduction of water is 20 minutes. The paste obtained is then introduced into the 1.5 mm multi-hole die. The spaghetti strings generated are smooth and break easily. They are dried in an oven at 60° C. for 18 hours. These dry spaghetti strings are then calcined at 400° C. then stages of 2 hours.

The extruded materials thus obtained after calcination have lengths which range between 3 and 20 mm.

### Comparative Example 3

### Preparation of a Catalyst C According to Patent FR 2 750 987

KMnO₄ (15.6 g; 95.9 mmol) is dissolved in water (240 mL) at ambient temperature in a 1-litre one-necked flask. Silica 60 (Merck, 240 g) is then added and the mixture is mechanically stirred for 2 hours. The water is then evaporated off under vacuum in a rotary evaporator (bath at 60° C.). The violet powder obtained is then gradually added to a vigorously stirred solution of MnSO₄—H₂O (37.2 g; 220.1 mmol) in water (400 mL). The mixture is stirred for three hours and the brownish solid is filtered off over sintered glass.

This solid is washed with water until the manganese ions (characterized by the formation of a precipitate by treatment with aqueous ammonia) have completely disappeared in the washing water. The solid is thoroughly spin-dried on the filter and is placed in Petri dishes; the thickness of the layers being 0.5 cm. Drying is carried out at 110° C. in a ventilated oven for hours. The fine brown powder thus obtained weighs 248 g.

### Example 4

### Hydration of 2-hydroxy-4-methylthiobutanenitrile to 2-hydroxy-4-methylthiobutanamide in the Presence of the Powder Catalysts A, B and C

This example gives the results of measuring the conversion of 2-hydroxy-4-methylthiobutanenitrile in the presence of the compositions of the previous examples and in the manner which follows.

5 g of compound according to one of the examples above are ground and screened so as to recover the particle size fraction between 100 and 200 μm.

0.6 g of this particle size fraction is introduced into a Schott tube. The reaction mixture, composed of a solution of 23% by weight of HMTBN in water, is introduced into the Schott tube containing the catalyst. A magnetic bar is then placed in the Schott tube and stirred so as to homogenize the reaction mixture.

The Schott tube thus loaded is then heated to 75° C. The initial reaction time is considered to be when the temperature of 75° C. has been reached.

After reaction for 60 minutes, the heating is stopped and the catalyst is extracted from the reaction medium by filtration. The composition of the filtrate is analysed by HPLC.

The conversion of the HMTBN at time t is calculated relative to the HMTBN initially introduced, and the selectivity for various reaction products, such as HMTBM and HMTBA, at time t is calculated relative to the amount of this product formed at time t and to the amount of HMTBN at time t.

The catalytic performances of the powder catalysts are given in table 1.

TABLE 1

| | Reaction | Conversion | Selectivity | Selectivity |
|---|---|---|---|---|
| Ground | time | of HMTBN | for HMTBM | for HMTBA |
| catalyst | (min) | (%) | (%) | (%) |
| A (invention) | 60 | 90 | 70 | 4 |
| B (invention) | 60 | 100 | 93 | 2 |
| C (comparative) | 60 | 13 | 64 | 2 |

It is seen from the results in table 1 that the compositions of the invention (A and B) have catalytic activities that are greater than the catalyst described in patent FR 2 750 987 (C). After reaction for 60 minutes at 75° C., without the addition of sulphuric acid, the HMTBN conversions are greater than 90% for the examples of the invention and their selectivities for HMTBM are greater than 70%, whereas the comparative catalyst (C) shows only 13% HMTBN conversion and 63% selectivity for HMTBM.

### Example 5

### Hydration of 2-hydroxy-4-methylthiobutyronitrile to 2-hydroxy-4-methylthiobutyramide in the Presence of Catalyst B

This example gives the results of measuring the conversion of 2-hydroxy-4-methylthiobutyronitrile in the presence of catalyst B over time and in the following way.

80 mL of catalyst B described in example 2 are introduced into a fixed-bed batch reactor with flow recirculation. 180 mL of industrial flow of HMTBN diluted in water so as to have 28% by weight of HMTBN in the reaction flow are introduced into the reactor. The reaction flow is circulated in the reactor with a circulation flow rate of 12 l/h. The reactor is brought to the temperature of 75° C. The initial reaction time is considered to be when the temperature of 75° C. has been reached. Samples of the flow are taken over the course of the reaction so as to monitor the progression of the reaction. The amounts taken are very small and it is considered that the flow volume remains constant throughout the reaction. The composition of the samples taken is determined by HPLC.

The conversion of HMTBN at time t is calculated relative to the HMTBN initially introduced, and the selectivity for HMTBM at time t is calculated relative to the amount of HMTBM at time t and to the amount of HMTBN converted at time t.

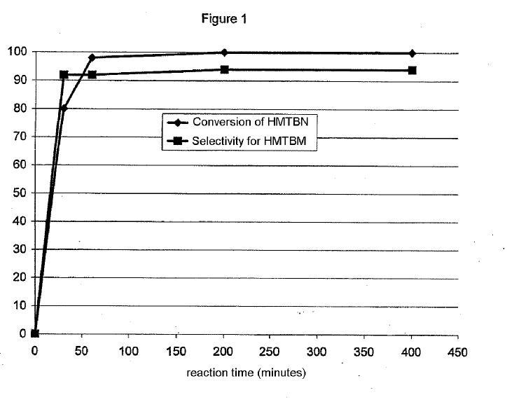

The catalytic performances of catalyst B over time are given in FIG. 1.

It is seen in FIG. 1 that catalyst B is very active with respect to the hydration of HMTBN and very selective for HMTBM. Furthermore, the HMTBM formed is very stable with time and does not decompose to HMTBA.

1. Process for the catalytic conversion of 2-hydroxy-4-methylthiobutanenitrile (HMTBN) to 2-hydroxy-4-methylthiobutanamide (HMTBM), in the presence of a solid catalyst comprising an active phase, wherein the catalyst is formulated and said conversion is carried out in a medium essentially free of strong mineral acid.

2. Process according to claim 1, wherein the active phase of said catalyst comprises at least one metal oxide comprises at least one of copper oxide, nickel oxide, iron oxide, zirconium oxide, manganese oxide and cerium oxide, and combinations of these oxides.

3. Process according to claim 1, wherein the catalyst is formulated in the presence of at least one diluent.

4. Process according to claim 3, wherein the diluent comprises at least one of zirconium oxide, titanium oxide, alumina, silica, clays such as bentonites, and attapulgite, and combinations thereof.

5. Process according to claim 1, wherein the proportion of the active phase is at least 30% (w/w) relative to the catalyst.

6. Process according to claim 3, wherein the proportion of the diluent is at most 70% (w/w) relative to the catalyst.

7. Process according to claim 1, wherein the catalyst is formulated by means of a first step comprising one of extrusion and wet granulation, and then a second step of heat treatment.

8. Process according to claim 7, wherein the formulating step is carried out with a binder which ensures cohesion between the active phase and the diluent.

9. Process according to claim 8, wherein the binder comprises one of water, natural polymers, organic polymers and sugars.

10. Process according to claim 7, wherein the heat treatment step is drying followed by calcination.

11. Process according to claim 1, wherein the conversion is carried out at a temperature which ranges from 0 to 100° C.

12. Process according to claim 1, wherein the duration of the conversion is greater than 45 minutes.

13. Process according to claim 1, wherein the HMTBN is in solution, at 20% to 80% by weight relative to the total weight.

14. Process according to claim 13, wherein the HMTBN is in solution in a solvent or a mixture of solvents comprising one of water and water-soluble solvents including acetone or methanol.

15. Process according to claim 1, wherein the HMTBN is present in a reaction medium from which it originates.

16. Process according to claim 15, wherein the HMTBN is obtained by reaction of hydrogen cyanide with 3-(methylthio)propionaldehyde (MTPA).

17. Process according to claim 15, wherein the HMTBN is obtained from acrolein and hydrogen cyanide, without isolation of intermediate products, and then addition of methylmercaptan (MSH).

18. Process for producing 2-hydroxy-4-methylthiobutanoic acid (HMTBA), comprising:
conversion of HMTBN to HMTBM carried out via a process as defined in claim 1, and
conversion of the HMTBM to HMTBA.

19. Process according to claim 18, wherein said conversion of the HMTBM to HMTBA is carried out in the presence of a catalyst based on one or more metal oxides.

20. Process according to claim 18, wherein said conversion of the HMTBM to HMTBA is carried out enzymatically, in the presence of an amidase.

21. Process according to claim 18, wherein said conversion of the HMTBM to HMTBA is carried out by hydrolysis of the HMTBM in the presence of a mineral acid.

22. Process according to claim 21, wherein the hydrolysis of the HMTBM is carried out in an aqueous solution, with sulphuric acid.

23. Process according to claim 18, wherein the HMTBA is obtained in the form of an ammonium salt, HMTBS.

24. Process according to claim 23, wherein the HMTBA is obtained from the ammonium salts via at least one of neutralization, electrodialysis and distillation.

25. Process according to claim 24, wherein the neutralization step is carried out on resins, or by acid neutralization.
